(19) United States
(12) Plant Patent Application Publication
(10) Pub. No.: US 2002/0104135 P1
(43) Pub. Date: Aug. 1, 2002

(54) Peach tree 'V75024'

(76) Inventor: Neil Miles, St. Catharines (CA)

Correspondence Address: FLYNN, THIEL, BOUTELL & TANIS, P.C., 2026 Rambling Road, Kalamazoo, MI, 49008-1699, US

(21) Appl. No.: 10/043,572

(22) Filed: Jan. 10, 2002

(30) Foreign Application Priority Data
Jan. 29, 2001 (CA) 01-2531

(51) Int. Cl. A01H 5/00

(52) U.S. Cl. PLT/197

(57) ABSTRACT

A new and distinct variety of peach tree, which has been given the designation ‘V75024’, is a high quality mid-to-late season non-melting fleshed clingstone peach that produces high quality mid-to late season fruit suitable for processing as a canned or frozen product.

### FIELD OF INVENTION

The present invention relates to a non-melting fleshed clingstone processing peach variety [Prunus persica (L.) Batsch] that is disease resistant, high quality, cold tolerant and productive.

### BACKGROUND OF THE INVENTION

‘V75024’ originated at the Horticultural Research Institute of Ontario (now the Department of Plant Agriculture, University of Guelph) at Vineland Station, Ontario, Canada. It arose as a single selection from the seedling population of V75024 that was derived from a controlled cross of ‘Suncling’ (patent status unknown) and ‘New Jersey Cling 81’ (patent status unknown) made in 1975. The selection was made in 1981 and then was asexually propagated by budding on peach seedling rootstock in the research nursery at Vineland Station. Comparative records of performance of ‘V75024’ have been kept on its performance at this site since that date. The asexual propagation demonstrates that such reproduction of the characteristics of the tree are consistent and are established or transmitted through succeeding generations. The tree of this variety is self-pollinating and self fruitful.

### SUMMARY OF THE INVENTION

The new and distinct non-melting fleshed clingstone processing peach, ‘V75024’, has distinguished itself as a processing peach. It is a high quality mid-to late season non-melting fleshed clingstone peach that produces high quality mid- to late season fruit suitable for processing as a canned or frozen product. It ripens 2 days later than ‘Babygold 5’ (patent status unknown), the variety it most closely resembles. Further compared to ‘Babygold 5’, ‘V75024’ has improved disease resistance, improved tree growth habit, good vigour, good tree and bud tolerance to low temperatures during the winter, good fruit productivity and large sized fruit. It has superior resistance to bacterial spot disease, Xanthimonus campestris pv pruni (Smith) Dye. It has acceptable tolerance to blossom blight and brown rot of stone fruits, both caused by Monolinia fructicola (G. Went) Honey and to Leucostoma canker caused by Leucostoma persoonii Hohn. and L. cincta (FR.:Fr.). The moderately firm flesh of the fruit is of suitable texture, sugar and acid characteristics such that the canned and frozen products meet or exceed industry standards.

### BRIEF DESCRIPTION OF THE DRAWINGS

FIG. 1 is a photographic illustration of the fruit of ‘V75024’ on the tree.

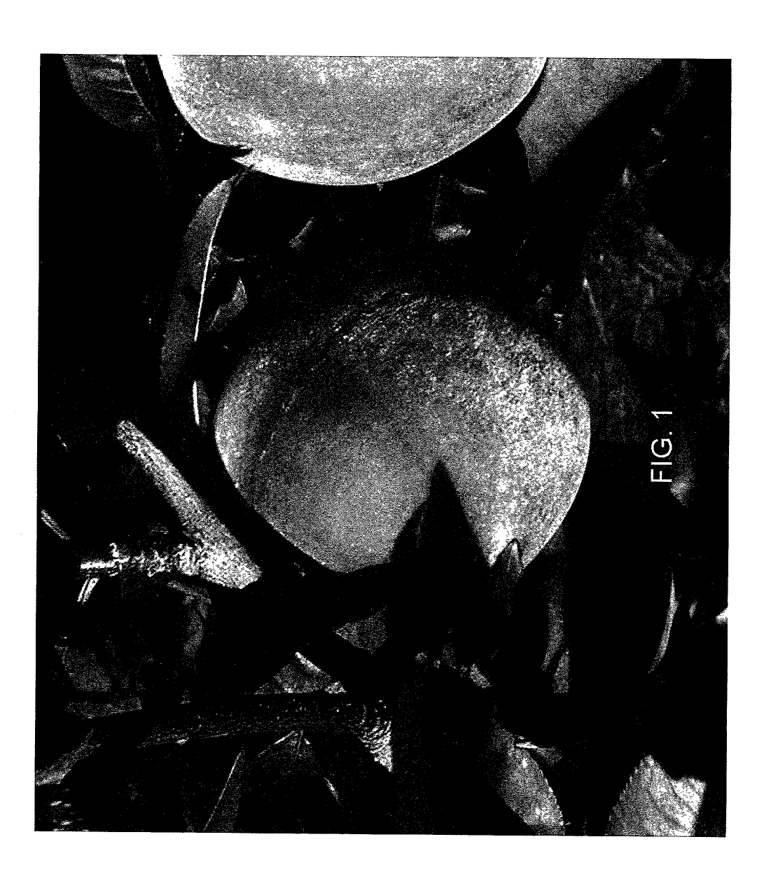

FIG. 2 is a photograph depicting the external and internal characteristics of the fruit of ‘V75024’.

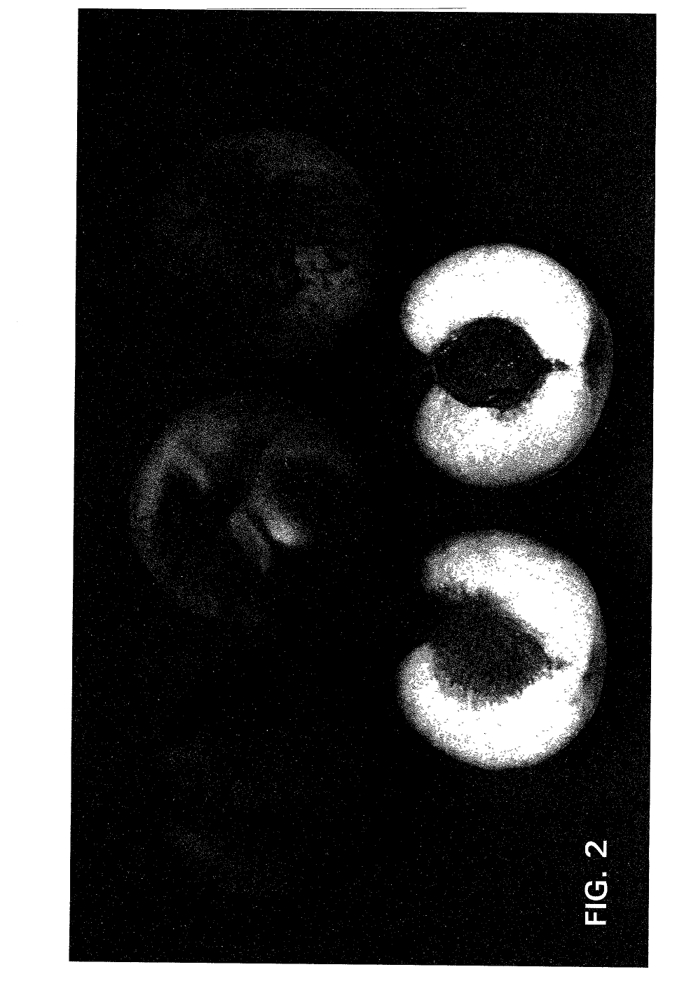

### DESCRIPTION OF VARIETY

‘V75024’ is a non-spur tree type of strong vigour similar to ‘Catherina’ (patent status unknown), ‘Babygold 5’ and ‘Babygold 7’(patent status unknown). The trees are semi-horizontal being intermediate between ‘Catherina’ which is horizontal and ‘Babygold 5’ and ‘Babygold 7’ which are semi-erect. The leaf blade is of medium size, with a mean length (10 leaves measured) of 144.4 mm, a range of 130-160 mm, a standard deviation of 10.44 mm. The mean width (10 leaves measured) of 37.8 mm, a range of 34-47 mm, a standard deviation of 3.97 mm. These measurements are comparable to ‘Catherina’ but less than for either ‘Babygold 5’ or ‘Babygold 7’. The leaf blade profile is flat comparable to ‘Catherina’ but dissimilar to the upfolded leaves of ‘Babygold 5’ and ‘Babygold 7’. The leaf blade tip is recurved downwards, the same as comparative varieties. The angle at the base of the leaf blade is acute compared to the right angle of ‘Catherina’ and the obtuse angle of ‘Babygold 5 and Babygold 7’. The angle at the tip of the leaf blade is acute compared to the right angle of ‘Babygold 7’. The anthocyanin coloration of the leaf blade observed in mid-summer is absent. On a scale of 1 (absent) and 9 (present) the serration of the leaf blade of ‘V75024’ is a 4 compared to 2 in ‘Catherina’, 6 in ‘Babygold 5’ and 4 in ‘Babygold 7’. The petiole length is rated as medium-long, longer than the comparative varieties. The nectaries are present on the petiole. The shape of nectaries on the petiole is kidney-shaped. The number of nectaries on the petiole is normally 2 as they are for ‘Babygold 5’ and ‘Babygold 7’ but there were more than 2 on the petioles of ‘Catherina’.

‘V75024’ anthocyanin coloration on the flowering shoot observed in mid-summer was present and of medium intensity. The density of flower buds observed in dormant period are medium-dense while the density of ‘Catherina’ was dense and ‘Babygold 5’ and ‘Babygold 7’ were medium. The distribution of flower buds observed in dormant period are in groups of 2 or more. The time of the beginning of flowering is medium-late in ‘V75024’ and comparative varieties. The flower shape is campanulate in ‘V75024’, ‘Babygold 5’ and ‘Babygold 7’ and rosaceous in ‘Catherina’. The calyx color is Burgandy in ‘V75024’, ‘Babygold 5’ and ‘Babygold 7’ and greenish yellow in ‘Catherina’. The petal shape is elongated in ‘V75024’, ‘Babygold 5’ and ‘Babygold 7’ and rounded in ‘Catherina’. The petal size is medium in ‘V75024’, ‘Babygold 5’ and ‘Babygold 7’ and large in ‘Catherina’. The petal color is medium pink in ‘V75024’ and the comparative varieties. Petal numbers are 5 and petals are lightly striped in ‘V75024’ and comparative varieties. All had one pistil per flower. The position of the stigma compared to the anthers is above in ‘V75024’, at the same level in ‘Catherina’ and slightly above in ‘Babygold 5’ and ‘Babygold 7’. Anthers in flowers in ‘V75024’ and all comparative varieties produced a moderate amount of pollen.

The time of fruit maturity for ‘V75024’ is late, medium in Catherina, medium-late in ‘Babygold 5’ and very late in ‘Babygold 7’. Fruit size is rated large for ‘V75024’, ‘Catherina’ and ‘Babygold 7’ and very large in ‘Babygold 5’. Fruit shape is rounded in ‘V75024’, ‘Catherina’, ‘Babygold 5’ and ovate in ‘Babygold 7’. The shape of the fruit tip is dimpled in all but ‘Babygold 7’ which is tipped. Fruit of all were symmetrical along the suture. ‘V75024’ had a more prominent suture than comparative varieties. The depth of the petiole cavity was medium-shallow for ‘V75024’, medium for ‘Catherina’ and ‘Babygold 7’ and shallow for ‘Babygold 5’. The width of the petiole cavity is medium-narrow for ‘V75024’ and ‘Catherina’ and narrow for ‘Babygold 5’ and ‘Babygold 7’. The fruit skin ground color is cream-yellow in ‘V75024’, orange-yellow in ‘Catherina’, and greenish-yellow in ‘Babygold 5’ and ‘Babygold 7’. The anthocyanin color of the skin of the fruit is dark red in ‘V75024’, orange red in ‘Catherina’ and pink-red in ‘Babygold 5’ and ‘Babygold 7’. The pubescence on the skin of the fruits of ‘V75024’ is medium-dense and medium-sparse in the comparative varieties. The flesh of all is non-melting with firmness of ‘V75024’ being firm, ‘Catherina’ medium-firm and ‘Babygold 5’ and ‘Babygold 7’ medium. The ground color of the flesh is yellow to orange-yellow. Fruit anthocyanin coloration at the pit is present in ‘V75024’, ‘Babygold 5’ and ‘Babygold 7’ but absent in ‘Catherina’.

The size of the stone of ‘V75024’ is medium and medium-small for ‘Catherina’ and medium-large for ‘Babygold 5’ and ‘Babygold 7’. The stone shape is ovoid for ‘V75024’, ‘Babygold 5’ and ‘Babygold 7’, but globular for ‘Catherina’. The stone color is red for ‘V75024’, ‘Babygold 5’ and ‘Babygold 7’, but tan for ‘Catherina’. The percentage of split or shattering of the pits of ‘V75024’ and all of the comparative varieties is absent or very low. The flesh adherence to the pit is present and all are considered clingstone varieties.

‘V75024’, ‘Babygold 5’ and ‘Babygold 7’ ‘Catherina’ all are moderately resistant to Leucostoma canker disease. ‘V75024’ and ‘Catherina’ are resistant to bacterial spot disease caused by Xanthimonus campestris pv pruni (Smith) Dye but ‘Babygold 5’ is susceptible and ‘Babygold 7’ is moderately susceptible.

I claim:

1. A new and distinct peach tree variety substantially as shown and described herein.
